(19) United States
(12) Patent Application Publication
(10) Pub. No.: US 2016/0038240 A1
(43) Pub. Date: Feb. 11, 2016

(54) IRRIGATION EQUIPMENT LIFTING APPARATUS

(71) Applicant: JOSE GUERRERO, Glenns Ferry, ID (US)

(72) Inventor: JOSE GUERRERO, Glenns Ferry, ID (US)

(21) Appl. No.: 14/456,576

(22) Filed: Aug. 11, 2014

(51) Int. Cl. A61B 19/00; B66F 11/00; B66F 3/00

(52) U.S. Cl. CPC A61B 19/26; B66F 3/00; B66F 11/00

(57) ABSTRACT

An irrigation equipment lifting apparatus includes a jack assembly. The irrigation equipment lifting apparatus further includes a lateral member mounted to and extending laterally from the jack assembly. The lateral member includes a concave circular depression defined therein.

### FIELD OF THE DISCLOSURE

The present disclosure relates to a lifting appara us and more particularly to an irrigation equipment lifting apparatus.

### BACKGROUND

Pivot-type irrigation systems include a central pivot, at least one irrigation pipe extending out from the central pivot, and one or more motorized wheeled towers. The motorized wheeled towers are coupled to the central pivot via the irrigation pipe such that as the motorized wheeled towers move, the pipe revolves around the central pivot. From time to time, a wheel of one of the wheeled towers may require maintenance, for example, due to a flat tire. A conventional jack system may be used in an attempt to lift a horizontal frame member (e.g., a pipe) of the wheeled tower to gain access to the wheel.

However, current jack systems used to lift irrigation equipment are not shaped to fit horizontal frame members of a wheeled tower. Because current jack systems do not fit the horizontal frame members, they may slip or otherwise become displaced during use placing the user in danger and potentially causing damage to the jack system and/or the irrigation system. Additionally, current jack systems are not adapted to be used in afield where the wheeled tower may be located, which may be muddy due to irrigation. A muddy field may not provide an adequate base upon which a conventional jack system may be used. Thus, an improved jack system for the lifting of irrigation equipment may be needed to overcome these or other shortcomings of current jack systems.

### SUMMARY

In an embodiment, an irrigation equipment lifting apparatus includes a jack assembly that may overcome some of the disadvantages discussed above. The apparatus further includes a lateral member mounted to and extending laterally from the jack assembly. The lateral member includes a concave circular depression defined therein.

In an embodiment, the concave circular depression has the shape of a half-pipe. A radius of the concave circular depression may be greater than at least 2 inches. The concave depression may be shaped to substantially fit a horizontal frame member of a wheeled tower associated with an irrigation system. For example, the irrigation system may be a pivot-type irrigation system.

In an embodiment, the irrigation equipment lifting apparatus further includes a diagonal support beam mounted to the jack assembly and to the lateral member.

In an embodiment, the irrigation equipment lifting apparatus also includes a handle mounted to the jack assembly.

In an embodiment, the jack assembly is a telescoping jack assembly and includes an outer tubing. The lateral member may be mounted to the outer tubing. The jack assembly may further include an inner tubing configured to reside at least partially inside the outer tubing.

In an embodiment, the jack assembly further includes a base mounted to the inner tubing. The jack assembly may also include a removable foot coupled to the base. A surface area of the removable foot may be greater than a surface area of the base.

In an embodiment, the jack assembly also includes a crank arm. Rotation of the crank arm may actuate a telescoping action between the outer tubing and the inner tubing. The jack assembly may further include a handle coupled to the crank arm. The handle facilitates rotation of the crank arm.

In an embodiment, the jack assembly also includes a housing cap mounted to the outer tubing. The outer tubing, the inner tubing, and the housing cap may define a cavity within the jack assembly.

In an embodiment, the lateral member is comprised of aluminum, steel, cast iron, or a combination thereof. The lateral member may be welded to the jack assembly.

In an embodiment, an irrigation equipment lifting apparatus includes an outer tubing. The irrigation equipment lifting apparatus further includes an inner tubing configured to reside at least partially inside the outer tubing. The irrigation equipment lifting apparatus also includes a lateral member mounted to and extending laterally from the outer tubing. The lateral member includes a concave circular depression defined therein.

In an embodiment, the irrigation equipment lifting apparatus further includes a crank arm. Rotation of the crank arm may actuate a telescoping action between the outer tubing and the inner tubing.

In an embodiment, the irrigation equipment lifting apparatus further includes a base mounted to the inner tubing and a removable foot attached to the base by an attachment system. The removable foot may have a larger surface area than the base.

In an embodiment, the irrigation equipment lifting apparatus also includes a diagonal support beam mounted to the outer tubing and to the lateral member. The diagonal support beam may enable the lateral member to support a weight associated with a wheeled tower of an irrigation system.

One or more of the embodiments described herein may overcome one or more disadvantages associated with traditional irrigation lifting systems. For example, one or more of the embodiments described herein may inhibit a horizontal frame member of an irrigation system from slipping or otherwise become displaced while being lifted. As another example, one or more of the embodiments described herein may be adapted from used in a field that is muddy or otherwise unstable due to irrigation. Other benefits and advantages of the embodiments described herein may be apparent to those of ordinary skill in the art.

### BRIEF DESCRIPTION OF THE DRAWINGS

FIG. 1 illustrates an embodiment of an irrigation lifting system;

FIG, 2 illustrates a cross-section of an embodiment of an irrigation lifting system;

FIG. 3 illustrates a pivot-type irrigation system compatible with an embodiment irrigation lifting system; and

FIG. 4 illustrates a close-up view of a portion of a pivot-type irrigation system being lifted by an embodiment of an irrigation lifting system.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the spirit and scope of the invention as defined by the appended claims.

### DETAILED DESCRIPTION

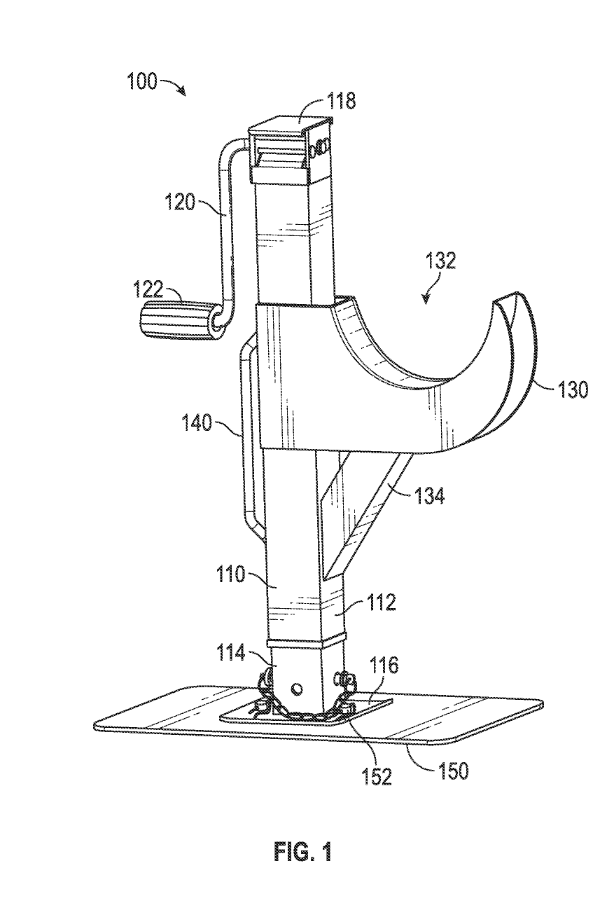

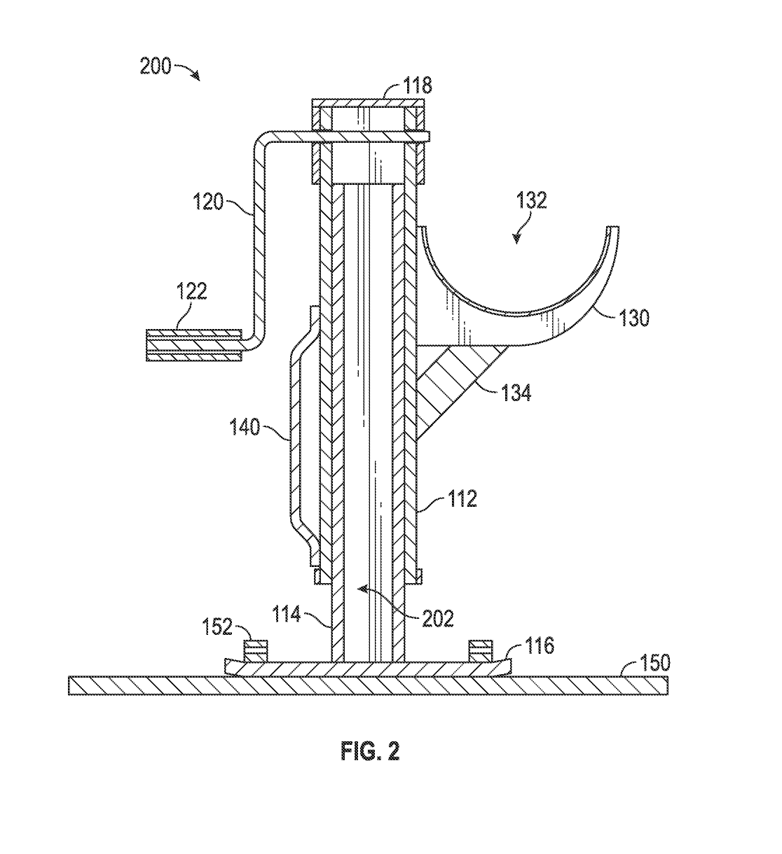

Referring to FIG. 1, an illustration of an embodiment of an irrigation lifting system 100 is depicted. The irrigation lifting system 100 may include a jack assembly 110, a lateral member 130, a handle 140, and a removable foot 150.

The jack assembly 110 may be a telescoping-type jack assembly. For example, the jack assembly 110 may include an outer tubing 112 and an inner tubing 114. A portion of the inner tubing 114 resides inside the outer tubing 112. During contraction, or lowering, of the jack assembly 110, the inner tubing 114 may slide (i.e., telescope) into to the outer tubing 112 to shorten the jack assembly 110. Conversely, during expansion, or raising, of the jack assembly 110, the inner tubing 114 may slide out of the outer tubing 112 to lengthen the jack assembly 110.

The jack assembly 110 may further include a base 116 and a housing cap 118. The base 116 may provide a platform for holding the jack assembly 110 upright. The base 116 may further couple the jack assembly 110 to a removable foot 150 as described herein.

The housing cap 118 may protect an inside of the jack assembly 110 from moisture and dirt. For example, the housing cap 118 may prevent dirt and moisture from entering the jack assembly 110. To illustrate, the inside of the jack assembly 110 may include various mechanisms (not shown) to perform functions such as telescoping as described herein. The various mechanisms may become damaged due to clogging and/or rust if exposed to dirt and/or moisture.

The jack assembly 110 may further include a crank arm 120 and a crank handle 122. Rotation of the crank arm 120 may actuate a telescoping action between the outer tubing 112 and the inner tubing 114. For example, the crank arm 120 may actuate various mechanisms internal to the jack assembly 110 to cause the jack to expand or contract. To illustrate, turning the crank arm 120 in a first direction may cause the jack assembly 110 to expand (increasing a distance between the base 116 and the housing cap 118). Turning the crank arm 120 in a second direction may cause the jack assembly 110 to contract (decreasing a distance between the base 116 and the housing cap 118). The various mechanisms may include levers, screws, hydraulic chambers, and other mechanisms usable to perform the functions described herein as. An example of various mechanisms usable to perform the functions described herein may be found at least in U.S. Pat. No. 8,523,148 filed Jul. 16, 2008 and entitled “Jack Assembly,” hereby incorporated by reference in its entirety. Another example of various mechanisms usable to perform the functions described herein may be found at least in U.S. Pat. No. 6,926,261 filed Jul. 26, 2004 and entitled “Trailer Tongue Jack,” hereby incorporated by reference in its entirety.

The crank handle 122 may facilitate the use of the crank arm 120. For example, the crank handle 122 may be free to rotate around a portion of the crank arm 120. Because the crank handle 122 is free to rotate, the crank handle 122 may enable a user of the irrigation lifting system 100 to grasp the crank handle 122 and turn the crank arm 120 without readjusting the user's grasp.

Although FIG. 1 illustrates the jack assembly 110 as a telescoping-type jack assembly, in other embodiments the jack assembly may be a scissor-type jack assembly, a screw-type jack assembly, a beam-type jack assembly (e.g. a farm jack), another type of jack assembly, or a combination thereof.

The lateral member 130 may be mounted to and may extend laterally from the jack assembly 110. For example, a portion of the lateral member may be coupled to the jack assembly using one or more fixation methods. In an embodiment, the lateral member 130 is welded to the jack assembly 110. In an embodiment, the lateral member 130 is bolted to the jack assembly 110. In an embodiment, the lateral member 130 is adhered to the jack assembly 110 using an industrial adhesive. In an embodiment, one or more of a welding process, a bolting process, and an adhesive process are used to attach the lateral member 130 to the jack assembly 110. Various mechanisms and/or methods may be used to adequately secure the lateral member 130 to the outer tube 112 of the jack assembly 110 as would be appreciated by one of ordinary skill in the art having the benefit of this disclosure. The lateral member 130 may be made of various materials that permit the raising of irrigation equipment. For example, the lateral member 130 may be, but is not limited to, aluminum, steel, cast iron, another material, or any combination thereof.

As illustrated in FIG. 1, the lateral member 130 may include an indentation shaped to substantially tit an outer surface of the jack assembly 110 (e.g., an outer surface of the outer tubing 112). For example, the lateral member 130 may be in contact with multiple sides of the jack assembly 110. When a heavy load is placed on the lateral member 130, the indentation may enable the irrigation lifting system 100 to relieve high tension areas at the junction of the lateral member 130 and the jack assembly 110. For example, a contact surface area between the lateral member 130 and the outer surface of the jack assembly 110 may be larger as compared to a lateral member that does not include an indentation. The larger contact surface area may enable the irrigation lifting system 100 to deflect a tension path between the jack assembly 110 and the lateral member 130. The tension path may be further deflected through the multiple sides of the jack assembly 110. Relieving the high tension areas may prevent the irrigation lifting system 100 from breaking at the junction of the lateral member 130 and the jack assembly 110.

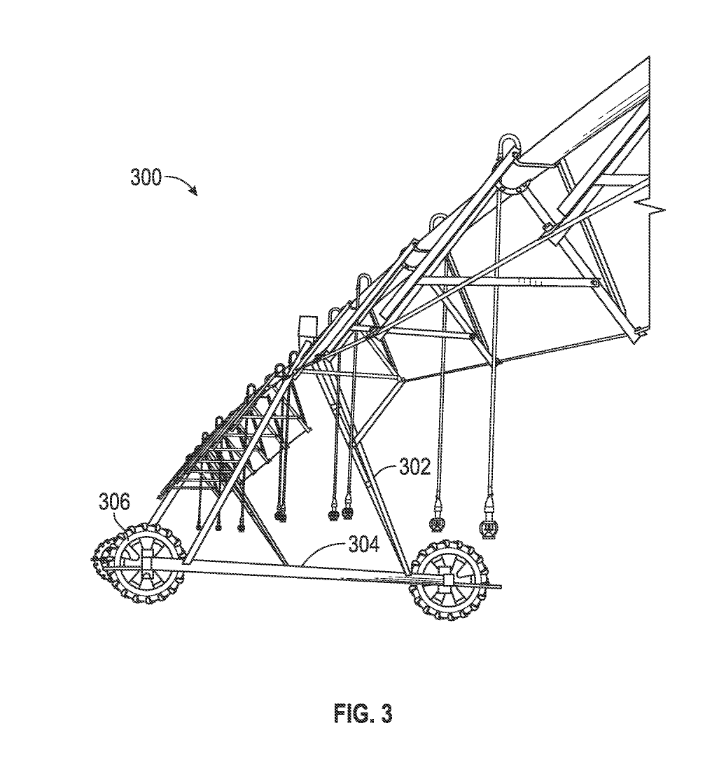

The lateral member 130 may include a concave circular depression 132 defined therein, The concave circular depression may have the shape of a half-pipe. For example, a profile of the concave circular depression 132 may be semi-circular such that a surface of the concave circular depression 132 substantially fits a surface of a pipe or other cylindrical or rounded object. The profile of the concave circular depression 132 may include less than a full circle to enable the cylindrical or rounded object to be inserted and removed from the concave circular depression 132 without great force and/or without damaging the lateral member 130. For example, the profile of the concave circular depression 132 may define less than or equal to 180 degrees of a circle. In an embodiment, the concave circular depression 132 is shaped to substantially fit a horizontal frame member of a wheeled tower associated with an irrigation system as described further with reference to FIG. 3. Some wheeled towers may include horizontal frame members that are cylindrical with a radius of about one inch. Hence, in an embodiment a radius of the concave circular depression 132 may be greater than or equal to at least one inch. Other wheeled towers may include horizontal frame members that are cylindrical with a radius of about two inches. Hence, in an embodiment a radius of the concave circular depression 132 may be greater than or equal to at least two inches.

The lateral member 130 may be supported by a diagonal support beam 134 mounted to the jack assembly 110 and to the lateral member 130. The diagonal support beam 134 may enable the irrigation lifting system 100 to further relieve high tension areas at the junction of the lateral member 130 and the jack assembly 110 by deflecting at least a portion of the stress placed on the junction between the jack assembly 110 and the lateral member 130. Hence, the diagonal support beam 134 may further help to prevent breakage at the junction of the lateral member 130 and the jack assembly 110. In an embodiment, the diagonal support beam 134 enables the lateral member 130 to support a weight associated with a wheeled tower of an irrigation system.

The handle 140 may facilitate movement and placement of the irrigation lifting system 100. For example, the handle 140 may enable a user of the irrigation lifting system 100 to hold and/or carry the irrigation lifting system 100 comfortably. The handle 140 may also facility placement of the irrigation lifting system 100 in a work position (e.g., underneath a horizontal frame member of a wheeled tower). Although FIG. 1 depicts the handle 140 as being attached to a side of the outer tubing 112, in other embodiments the handle may be attached to one or more other portions of the jack assembly 110.

The removable foot 150 may be attached to and removable from the jack assembly 110. For example, the removable foot 150 may be coupled to the base 116 of the jack assembly 110 by an attachment system 152. As shown if FIG. 1, the attachment system 152 may include one or more shafts protruding from the removable foot 150. The one or more shafts may be inserted into and protrude from the base 116. A pin may be inserted into the one or more shafts to lock the removable foot 150 in place relative to the base 116. The removable foot may have a larger surface area than the base 116 and may thereby provide greater support for the irrigation lifting system 100 on unstable terrain than the base 116. For example, in cases where the irrigation lifting system 100 may be used on uneven, wet, and/or soft ground (e.g., a cultivated field), the removable foot 150 may steady the irrigation lifting system 100 and prevent the irrigation lifting system 100 from sinking into the ground as a heavy load is place on the irrigation lifting system 100. However, the larger removable foot 150 may be removed from the base 116 when not needed to increase the mobility of the irrigation lifting system 100.

During operation, a user of the irrigation lifting system 100 may grasp the handle 140 to place the irrigation lifting system 100 at with the removable foot 150 in contact with the ground and with the lateral member 130 underneath a horizontal frame member of a wheeled tower of a pivot-type irrigation system. The user may use the crank handle 122 to turn the crank arm 120 causing the jack assembly 110 to expand. The expansion of the jack assembly 110 may cause a distance between the lateral member 130 and the removable foot 150 to increase. As the jack assembly 110 expands, the horizontal frame member may be received at the concave circular depression 132 and may be lifted from the ground by the lateral member 130.

A benefit of using the irrigation lifting system 100 to lift a horizontal frame member from the ground is that the irrigation lifting system 100 is less likely to slip underneath the horizontal frame member as compared to jack systems that do not include the lateral member 130 with the concave circular depression 132. Further, the removable foot 150 may provide more stability during lifting as compared to jack systems that do not include the removable foot 150. As such, the irrigation lifting system 100 may be more suitable for lifting wheeled towers of pivot-type irrigation systems.

Referring to FIG. 2, a cross-sectional illustration of an embodiment of an irrigation lifting system 200 is depicted. The irrigation lifting system 200 may include the outer tube 112, the inner tube 114, the base 116, the housing cap 118, the crank arm 120, the crank handle 122, the lateral member 130, the circular depression 132, the diagonal support beam 134, the handle 140, the removable foot 150, and the attachment system 152. The outer tube 102, the inner tube 104, the base 116, and the housing cap 118 may form a cavity 202. The cavity 202 may be protected from dirt and moisture. Although FIG. 2 illustrates the cavity 202 as being empty, other embodiments may include one or more mechanisms within the cavity 202 to enable the jack to telescope as described herein.

Referring to FIG. 3, a pivot-type irrigation system 300 compatible with an embodiment of an irrigation lifting system is depicted. The pivot-type irrigation system 300 may include a wheeled tower 302 coupled to a center pivot via a pipe 308 extending radially from the center pivot, such that the wheeled tower 302 is configured to revolve around the center pivot. The wheeled tower 302 may include a horizontal frame member 304 and at least one wheel 306. The horizontal frame member 304 may be pipe shaped and have a radius of at least 2 inches.

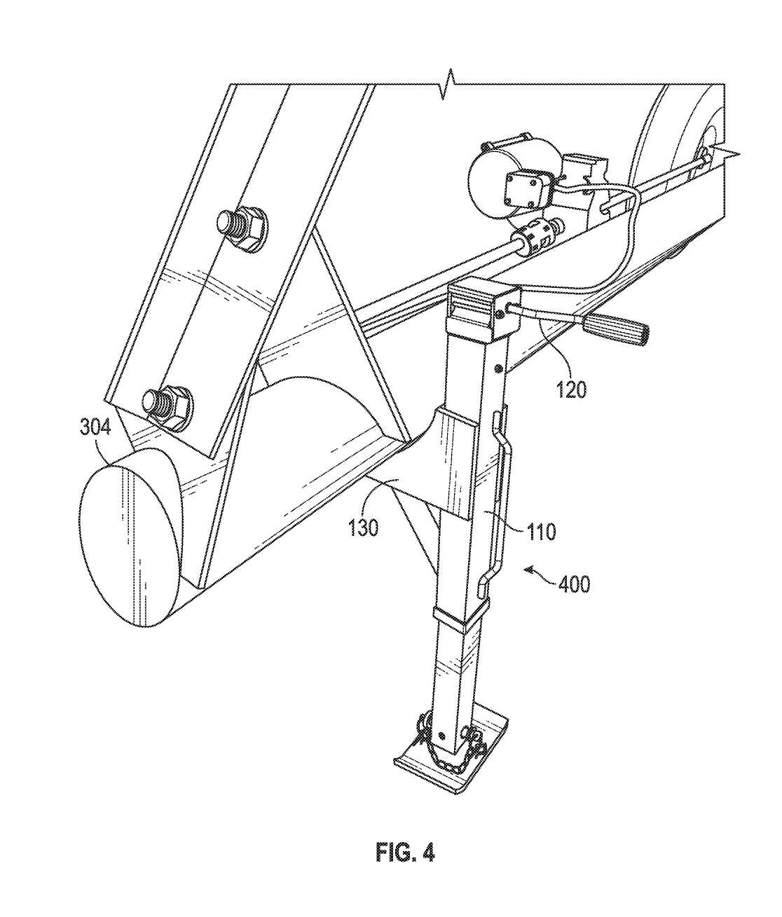

Referring to FIG. 4 a close-up view of a portion of a pivot-type irrigation system being lifted by an embodiment of an irrigation lifting system 400 is depicted. The irrigation lifting system 400 may include the jack assembly 110, the crank 120, and the lateral member 130. As shown in FIG. 4, the irrigation lifting system 400 may be used to lift the horizontal frame member 304, thereby also lifting the wheel 306. For example, a user may turn the crank arm 120 to expand the jack assembly 110. As the jack assembly 110 expands, the lateral member 130 may receive the horizontal frame member 304, lifting it relative to the ground. The lateral member 130 may have a shape that fits the horizontal frame member 304. For example, the lateral member 130 may be shaped like a half-pipe with a radius of at least 2 inches. Because the wheel 306 is attached to the horizontal frame member 304, the wheel 306 may be lifted as well.

By lifting the wheel 306, the irrigation lifting system 400 may enable a user to perform maintenance on the wheel 306. For example, the user may be able to replace a tire and/or perform other repairs that require the wheel 306 to be elevated. The shape of the lateral member 130 may inhibit the horizontal frame member 304 from slipping because the lateral member 130 is shaped to substantially fit the horizontal frame member 304 as described herein. Thus, the horizontal frame member 304 may not slip or otherwise become displaced relative to the irrigation lifting system 400 during use. In that way, the irrigation lifting system 400 may prevent potential injury to the user and prevent damage to the irrigation lifting system 400 and/or the wheeled tower 302.

Although various embodiments have been shown and described, the present disclosure is not so limited and will be understood to include all such modifications and variations are would be apparent to one skilled in the art.

What is claimed is:

1. An irrigation equipment lifting apparatus comprising:
a jack assembly; and
a lateral member mounted to and extending laterally from the jack assembly, the lateral member including a concave circular depression defined therein.

2. The apparatus of claim 1, wherein the concave circular depression has the shape of a half-pipe.

3. The apparatus of claim 1, wherein a radius of the concave circular depression is greater than at least 2 inches.

4. The apparatus of claim 1, wherein the concave depression is shaped to substantially fit a horizontal frame member of a wheeled tower associated with an irrigation system.

5. The apparatus of claim 4, wherein the irrigation system is a pivot-type irrigation system.

6. The apparatus of claim 1, further comprising a diagonal support beam mounted to the jack assembly and to the lateral member.

7. The apparatus of claim 1, further comprising a handle mounted to the jack assembly.

8. The apparatus of claim 1, wherein the jack assembly is a telescoping jack assembly.

9. The apparatus of claim 1, wherein the jack assembly comprises:
an outer tubing, wherein the lateral member is mounted to the outer tubing; and
inner tubing configured to reside at least partially inside the outer tubing.

10. The apparatus of claim 9, wherein the jack assembly further comprises a base mounted to the inner tubing.

11. The apparatus of claim 10, further comprising a removable foot coupled to the base, wherein a surface area of the removable foot is greater than a surface area of the base.

12. The apparatus of claim 9, wherein the jack assembly further comprises a crank arm, wherein rotation of the crank arm actuates a telescoping action between the outer tubing and the inner tubing.

13. The apparatus of claim 9, wherein the jack assembly further comprises a handle coupled to the crank arm, wherein the handle facilitates rotation of the crank arm.

14. The apparatus of claim 9, wherein the jack assembly further comprises a housing cap mounted to the outer tubing, wherein the outer tubing, the inner tubing, and the housing cap define a cavity within the jack assembly.

15. The apparatus of claim 1, wherein the lateral member is comprised of aluminum, steel, cast iron, or any combination thereof.

16. The apparatus of claim 1, wherein the lateral member is welded to the jack assembly.

17. An irrigation equipment lifting apparatus comprising:
an outer tubing;
an inner tubing configured to reside at least partially inside the outer tubing;
a lateral member mounted to and extending laterally from the outer tubing, the lateral member including a concave circular depression defined therein.

18. The apparatus of claim 17, further comprising a crank arm, wherein rotation of the crank arm actuates a telescoping action between the outer tubing and the inner tubing.

19. The apparatus of claim 17, further comprising a base mounted to the inner tubing and a removable foot attached to the base by an attachment system, wherein the removable foot has a larger surface area than the base.

20. The apparatus of claim 17, further comprising a diagonal support beam mounted to the outer tubing and to the lateral member, wherein the diagonal support beam enables the lateral member to support a weight associated with a wheeled tower of an irrigation system.
